(19) United States
(12) Patent Application Publication
(10) Pub. No.: US 2016/0287882 A1
(43) Pub. Date: Oct. 6, 2016

(54) FEEDTHROUGH DEVICE ESPECIALLY FOR A MEDICAL IMPLANT SYSTEM AND PRODUCTION METHOD

(71) Applicant: COMMISSARIAT A L'ENERGIE ATOMIQUE ET AUX ENERGIES ALTERNATIVES, Paris (FR)

(72) Inventors: Nicolas Karst, Folkling (FR); Fabrice Emieux, Voreppe (FR); Simon Perraud, Bandol (FR)

(73) Assignee: COMMISSARIAT A L'ENERGIE ATOMIQUE ET AUX ENERGIES ALTERNATIVES, Paris (FR)

(21) Appl. No.: 15/036,392

(22) PCT Filed: Nov. 12, 2014
(86) PCT No.: PCT/IB2014/065985

§ 371 Date: May 12, 2016

(30) Foreign Application Priority Data
Nov. 14, 2013 (FR) 1361131

(51) Int. Cl. A61N 1/375; H02G 15/013

(52) U.S. Cl. CPC A61N 1/3754; H02G 15/013

(57) ABSTRACT

The invention relates to a feedthrough device especially for a medical implant system, comprising: a flexible substrate locally comprising at least one means for stiffening the substrate, said at least one stiffening means having a through-opening; and at least one hermetic feedthrough comprising an electrical connection element, said feedthrough being hermetically joined to a stiffening means, such that said electrical connection element passes through the through-opening.

The invention relates to the technical field of implantable medical systems.

These systems consist of a battery and of a set of electronic components.

In order to avoid any contact between the electronic components and the biological tissues or fluids, hermetic casings are used for encapsulating them.

Feedthroughs are also used for allowing transport of electricity from the inside towards the outside of the casing.

In addition to the volume occupied by the electronic components and the battery, a non-negligible volume is occupied by the feedthroughs.

These feedthroughs are made, either by brazing platinum pins to a ceramic, or by co-sintering these pins with a green ceramic. An additional brazing step gives the possibility of binding the obtained element to a metal ferrule which will then be welded to the metal casing ensuring encapsulation.

A ceramic/metal feedthrough is for example described in document EP 1 107 264.

In order to ensure sufficient mechanical strength, it is difficult to reduce to below 1 mm the thickness of the ceramic use for making the feedthroughs, and shown in document U.S. Pat. No. 7,989,080.

Another method giving the possibility of making ceramic/metal feedthroughs consist of depositing thin layers in a biocompatible metal on a first green ceramic which will be co-sintered with a second green ceramic, as shown in document EP 0 660 449.

This method has the advantage of reducing the diameter of the hermetic feedthroughs and therefore their bulkiness, by the use of thin layers, and by thereby increasing the density thereof.

Document U.S. Pat. No. 5,750,926, as for it describes an encapsulation casing, one wall of which is made with the ceramics of the feedthroughs, these ceramics being generally biocompatible.

However, in order to ensure sufficient mechanical strength, it is necessary that the thickness of these ceramics be relatively significant, the corresponding wall of the casing is therefore rigid.

Thus, known implantable casings have significant rigidity and consequently can only be implanted in a limited number of localizations in the human body.

This is why the object of the invention is to reduce the thickness of implantable medical systems and to make these systems flexible so that they may be conformed at different implantation areas.

This allows implantation of these systems as close as possible to the treatment areas.

Thus, the invention relates to a feedthrough device especially for a medical implant system comprising:
a flexible substrate locally comprising at least one means for stiffening the substrate, said at least one stiffening means having a through-opening and
at least one hermetic feedthrough comprising an electrical connection element, said feedthrough being hermetically assembled to a stiffening means, so that said electrical connection element passes through the through-opening.

Thus, the substrate remains globally flexible in spite of the presence of the stiffening means, the latter allowing limitation of the mechanical stresses at the assembling between the substrate and the feedthroughs and the obtained feedthrough device is hermetic.

Preferably, said substrate consists in a metal sheet.

The stiffening means advantageously consists in a protruding portion with respect to the surface of the substrate.

The protruding portion may be a bulge or an over-thickness.

The feedthrough device according to the invention advantageously comprises at least one brazing joint between a feedthrough and a stiffening means.

This brazing joint may have a three-dimensional structure.

In a particular embodiment, the feedthrough has a T-shape, another brazing joint being present between an outgrowth and the substrate.

The invention also relates to a method for making a feedthrough device, comprising the following steps:
(a) producing a flexible substrate with at least one stiffening means and at least one through-opening in said at least one stiffening means,
(b) providing at least one hermetic feedthrough including an electrical connection element,
(c) hermetically assembling a feedthrough to said at least one stiffening means, so that the electrical connection element of said feedthrough partly passes into said through-opening.

The through-opening may be made before or after forming the stiffening means with which it is associated.

Preferably, the step (a) consists of producing at least one protruding portion with respect to the substrate.

In a first alternative, the step (a) consists in a local deformation of the substrate in order to form at least one bulge.

In a second alternative, the step (a) consists in providing material for locally forming at least one over-thickness.

In a third alternative, the step (a) consists in removing of material in at least two areas of a substrate in order to form at least one over-thickness between these areas.

Step (c) is advantageously achieved by means of at least one brazing joint.

In this case, it may be advantageous to position, between step (b) and step (c), a ceramic ring against said at least one stiffening means and on the substrate-holder side.

This gives the possibility of avoiding brazing of the substrate holder during the formation of the brazing joint.

The production method according to the invention may comprise, after step (c), an additional step consisting of depositing a polymeric material, in said at least one through-opening.

The invention also relates to a hermetic casing including a feedthrough device according to the invention and a lid hermetically sealed to the feedthrough device, notably intended for encapsulation of a medical device.

The medical implant system obtained will be flexible, by means of the flexibility of the feedthrough device. It will also be of a small thickness. This will facilitate its implantation in various areas of the body.

The invention will be better understood and other objects, advantages and features thereof will become more clearly apparent upon reading the description which follows and which is made with reference to the appended drawings, wherein:

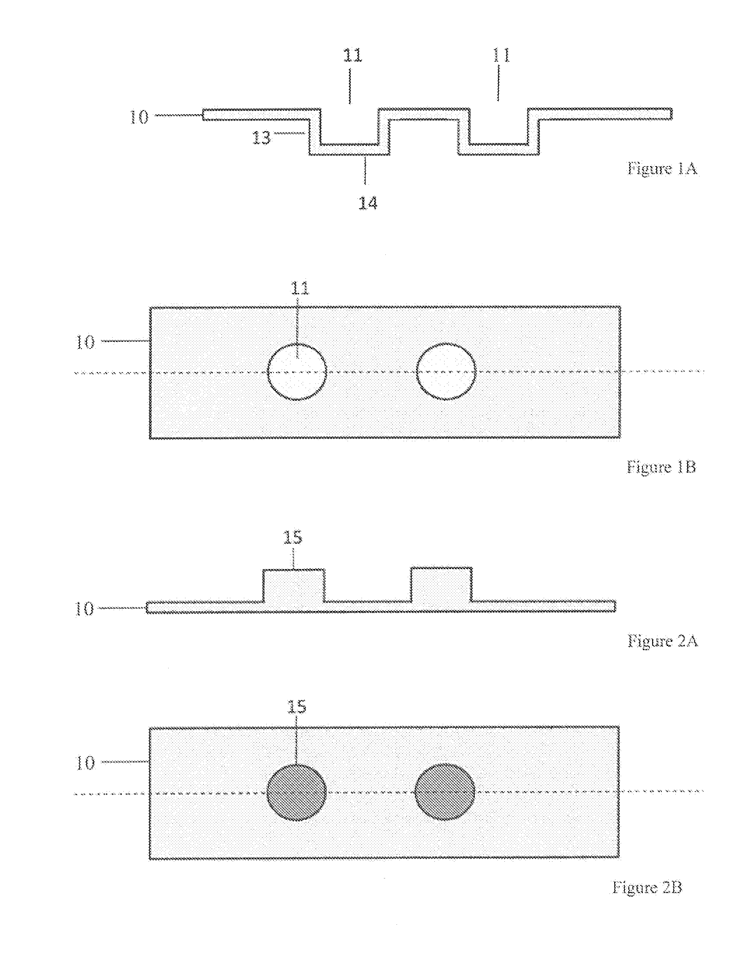

FIG. 1 (1A-1B) illustrates a first step of the method according to the invention wherein the stiffening means are made on the substrate of the encapsulation casing,

FIG. 2 (2A-2B) illustrates an alternative embodiment of the first step of the method according to the invention,

FIG. 3 (3A-3B) illustrates a second step of the method according to the invention, wherein an opening is made at the stiffening means, for the structure illustrated in FIG. 1,

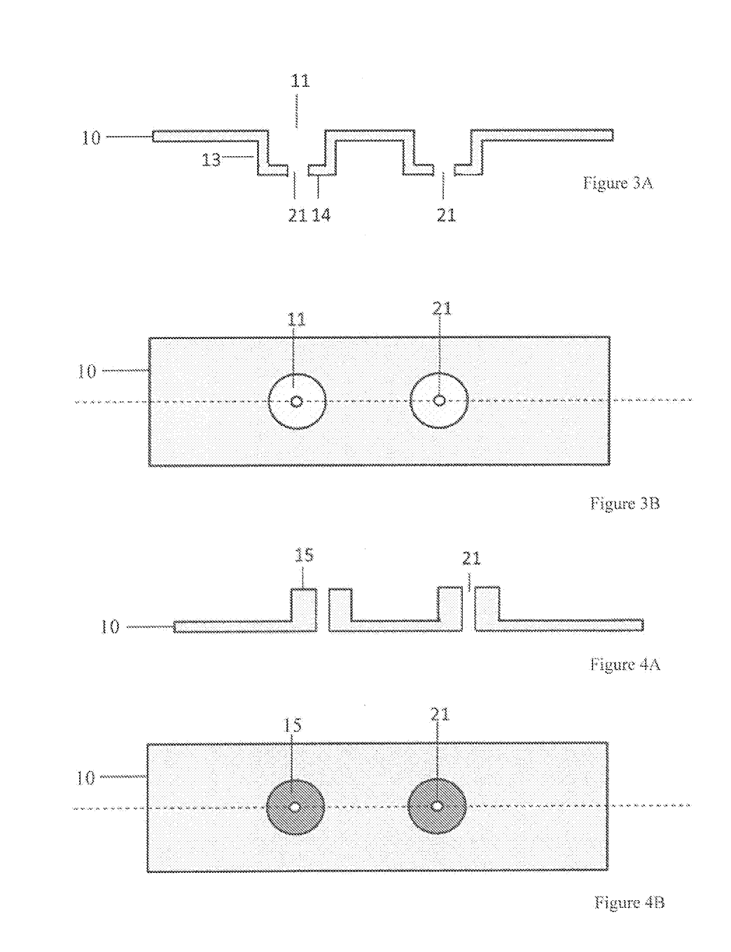

FIG. 4 (4A-4B) illustrates this second step of the method according to the invention for the structure illustrated in FIG. 2,

FIG. 5 (5A-5B) illustrates two alternative embodiments of a feedthrough,

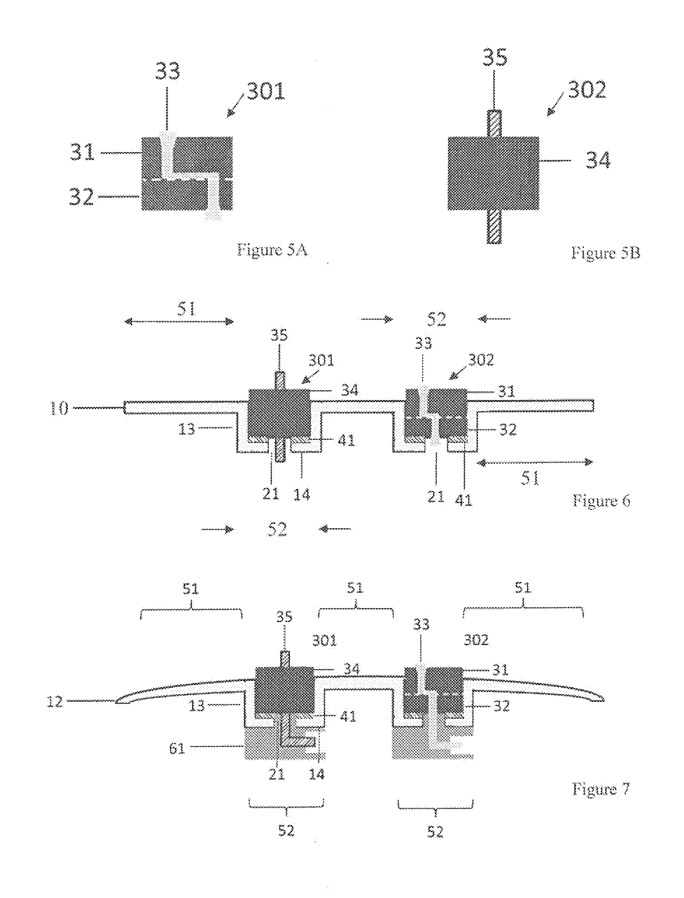

FIG. 6 illustrates a third step of the method according to the invention, wherein feedthroughs are assembled on the substrate illustrated in FIG. 3,

FIG. 7 is a sectional view, similar to FIG. 6, illustrating another step of the method according to the invention,

FIG. 8 is a sectional view illustrating a last step of the method according to the invention, wherein a lid is hermetically sealed on the substrate illustrated in FIG. 6,

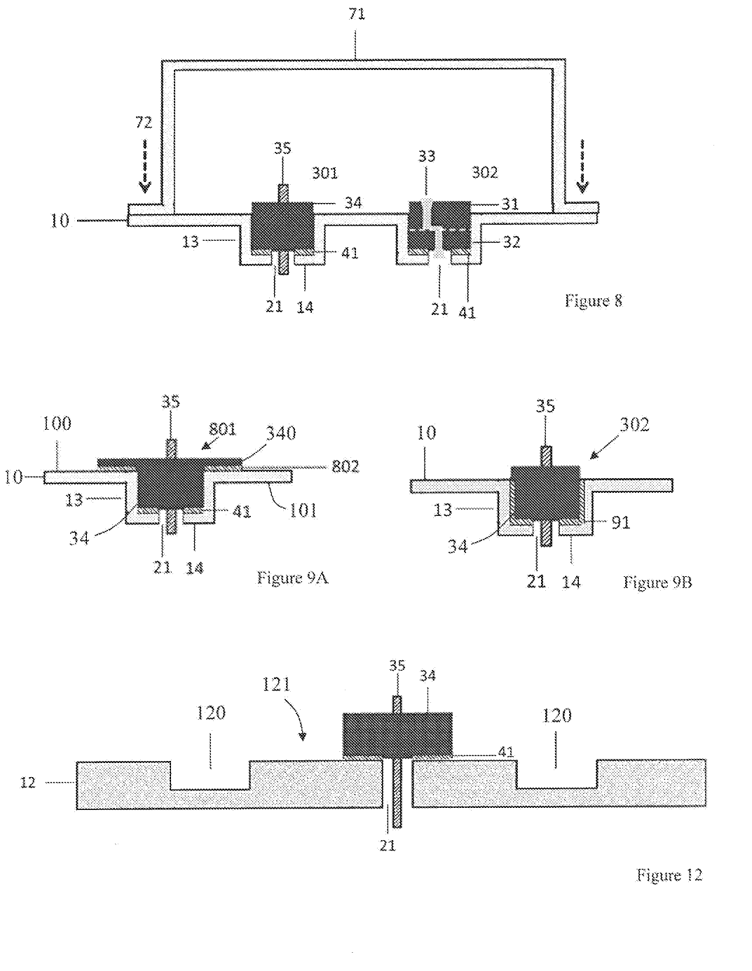

FIG. 9 (9A-9B) shows sectional views illustrating alternative embodiments of the step of the method illustrated in FIG. 6,

FIG. 10 (10A-10C) shows sectional views illustrating various alternatives of the substrate illustrated in FIG. 3,

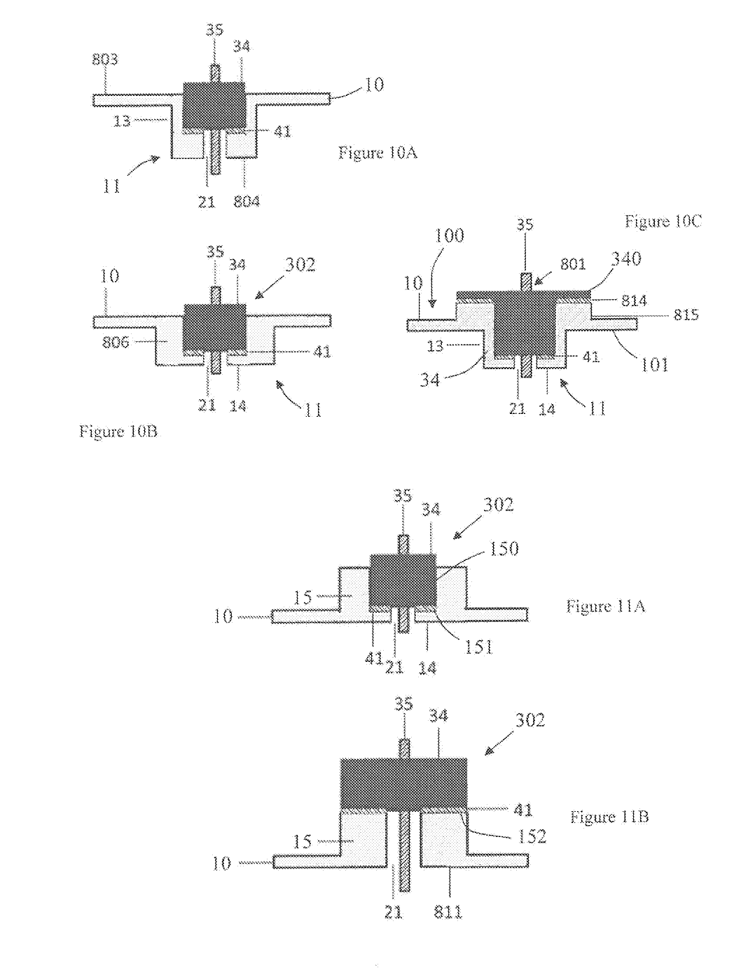

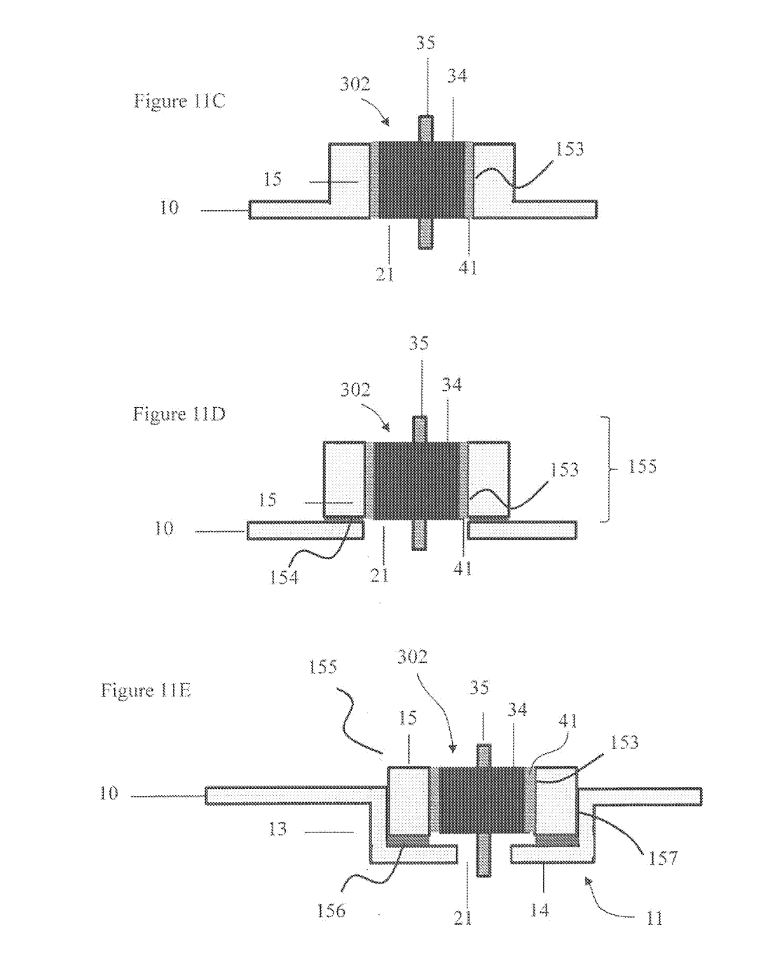

FIG. 11 (11A-11E) shows sectional views illustrating alternative embodiments of the third step of the method, notably carried out on the substrate illustrated in FIG. 4,

FIG. 12 is a sectional view illustrating an alternative of the method according to the invention and

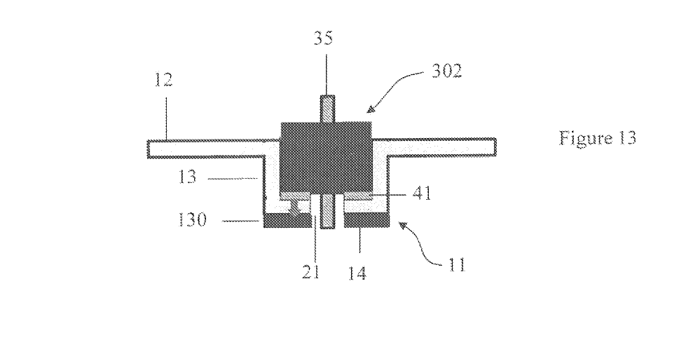

FIG. 13 is a sectional view illustrating an alternative of the method according to the invention, giving the possibility of avoiding brazing risks of the substrate holder.

The elements common to the various figures will be designated with the same references.

The method according to the invention is designed for making an encapsulation casing which is flexible or further conformable, this casing including hermetic feedthroughs giving the possibility of establishing electrical connections between the inside and the outside of this casing.

For this, the method according to the invention consists of making a feedthrough device both thin and flexible, i.e. capable to be conformed to a diameter comprised between 3 and 12 cm.

The first step of this method consists of locally making on a substrate, means for stiffening the substrate, in which feedthroughs will subsequently be assembled.

Thus, FIG. 1A is a sectional view of a substrate 10 in which two localized protruding portions or bulges 11 have been made.

FIG. 1B is a top view of the substrate illustrated in FIG. 1A.

The substrate 10 preferably appears as a sheet of small thickness, notably comprised between 20 and 250 μm.

Advantageously this is a flexible substrate.

Generally, the substrate will be made in a biocompatible material. Mention may notably be made of metal materials, such as titanium or a titanium, aluminium and vanadium alloy, such as TA6V or Ti-6Al-4V, or further a stainless steel, such as SS316L.

In practice, the substrate may be made in a ceramic material. However, such a material is generally not retained because the substrate should then have a relatively large thickness so as to be mechanically resistant and it is then non-flexible.

The bulges 11 may be obtained by deformation of the substrate and by various techniques known to one skilled in the art, such as for example stamping or hydroforming.

Preferably, the spacing between two bulges 11 is comprised between 0.1 mm and 5 cm and it is advantageously of the order of 0.1 mm.

Moreover, FIG. 1 shows two identical bulges. However, the dimensions of the bulges may be different, both as regards their depth and their diameter notably.

Advantageously, the depth of the bulges 11 will not exceed twenty times the thickness of the metal substrate 10 and will be preferentially equal to ten times the thickness of the metal substrate 10.

Thus, for a metal substrate 10 with a thickness of 50 μm, bulges 11 with a depth of about 500 μm will be preferred.

The bulges 11 will advantageously be of a circular shape but other shapes may be contemplated (square or pyramidal shape).

Advantageously, the diameter of the bulges 11 will be comprised between 500 μm and 2 cm, and preferentially equal to 3 mm.

A bulge 11 consists of a side wall 13 substantially perpendicular to the plane of the metal substrate 10 and of a base 14 substantially parallel to the plane of the metal substrate 10.

The side wall and the base have a thickness substantially identical with that of the substrate.

In certain particular cases, the angle between the wall 13 and the base 14 of the bulge 11 will be greater than 90°.

This may notably be the case when the bulges 11 are obtained by stamping. The bulges 11 then have a conical structure, which will allow feedthroughs which are themselves conical to be introduced therein.

This has an advantage when the brazing joint has a three-dimensional shape, as this will be illustrated in connection with FIG. 9B.

The base 14 in certain cases may have a concave structure. This may notably be advantageous for reducing the risks of inflammation of biological tissues, once the device is implanted.

Reference is now made to FIGS. 2A and 2B, FIG. 2A being a sectional view of the substrate 10 on which were made stiffening means consisting in localized over-thicknesses.

FIG. 2B is a top view of the substrate 10, with two protruding portions 15 formed by a localized over-thickness.

These over-thicknesses may be obtained by various techniques known to one skilled in the art, notably mechanical micro-machining or laser etching which give the possibility of removing material on a thick substrate. This embodiment is more particularly illustrated in FIG. 12.

These over-thicknesses may also be obtained by providing material. For example they may be assembled on the substrate 10 by laser welding.

Advantageously, the height of the over-thicknesses 15 will not exceed 20 times the thickness of the remainder of the substrate 10 and will preferentially be equal to about 10 times the thickness of the substrate 10.

Thus, for a substrate 10 having a thickness of 50 μm, the over-thicknesses 15 will have a height of about 500 μm.

The over-thicknesses 15 may appear in different shapes and will advantageously be circular.

In certain particular cases, these over-thicknesses 15 may have a conical structure.

It should be noted that, on a same substrate 10, the dimensions of the over-thicknesses may vary (diameter, thickness).

FIGS. 3 and 4 illustrate another step of the method in which the through-openings are made in the stiffening means made on the substrate.

Thus, FIG. 3A is a sectional view of the substrate illustrated in FIG. 1, FIG. 3B is a top view of FIG. 3A.

They show that through-openings 21 have been made in the bulges 11 and more particularly in the base 14 of these bulges.

FIG. 4A is a sectional view illustrating the substrate of FIG. 2, wherein through-openings 21 were made at the over-thicknesses 15.

These openings 21 may be obtained with different methods, such as for example machining methods.

In the examples illustrated in FIGS. 3 and 4, the openings 21 are substantially central. However, the invention is not limited to this embodiment and the openings 21 may be off-center.

Advantageously, the openings 21 will have a circular shape. They may also have a square, rectangular or conical shape.

Only one aperture 21 per bulge 11 or per over-thickness 15 is illustrated in FIGS. 3 and 4. However, several openings 21 per bulge 11 or per over-thickness 15 may be provided.

As regards the substrate illustrated in FIGS. 3A and 3B, the surface occupied by each opening 21 will advantageously be much smaller than the surface of the base 14.

Thus, in this example, the diameter of the opening 21 has to be much smaller than the diameter of the bulge 11.

This gives the possibility of mechanically protecting the hermetic feedthroughs, even when the ceramic thickness used in the making of these hermetic feedthroughs is small. Thus, the diameter of the opening 21 will advantageously be comprised between two and ten times the diameter of the electrical connection element of a feedthrough.

It will be preferentially twice larger than the diameter of the electrical connection element. As an example, the diameter of the pin 35 of the feedthrough illustrated in FIG. 5B is comprised between 50 μm and 1 mm and preferably equal to 100 μm.

Generally, the diameter of the opening 21 will also be much smaller than the diameter of the ceramic body of the hermetic feedthrough which will be assembled in the bulge 11.

It should be noted that the openings 21 may also be made prior to the step for forming the bulges 11 or the over-thicknesses 15.

Thus, these apertures may be made before stamping the substrate giving the possibility of forming bulges or before the welding on the substrate of over-thicknesses appearing as a recessed cylinder.

FIGS. 5A and 5B are sectional views illustrating ceramic/metal feedthroughs.

These feedthroughs are obtained with conventional techniques, notably those shown in documents U.S. Pat. No. 5,750,926 and EP-1 107 264.

Thus, the hermetic feedthrough 301 shown in FIG. 5A is obtained by producing a through-via within a first body 31 in non-sintered ceramic which is filled with an ink based on a biocompatible metal in order to produce a metal track 33. It is notably possible to use the following metals: Nb, Ta, Ti, Pt, Ir, Zr, Hf or Pt/Ir or Ir/Ta alloys for example.

The same step is carried out on a second body 32 in non-sintered ceramic.

A metal track 33 is then deposited on the upper face of the body 32 which will be put into contact with the lower face of the body 31. The contact area between the lower face of the body 31 and the upper face of the body 32 is schematically illustrated by the dotted line in FIG. 5A.

The assembly is co-sintered at a high temperature, thus giving the possibility of obtaining a hermetic feedthrough 301 ensuring electric continuity between the upper face of the body 31 and the lower face of the body 32. By simplification, there is no distinction in terms of numbering between the non-sintered ceramics and the sintered ceramics.

The hermetic feedthrough 302 shown in FIG. 5B is obtained by producing a through-via within a body 34 in non-sintered ceramic in which a metal pin 35 (for example in platinum) will be positioned.

The assembly is co-sintered at a high temperature thereby giving the possibility of obtaining a hermetic feedthrough 302 ensuring the electric continuity between the upper face of the body 34 and the lower face of the body 34. By simplification, there is no distinction in terms of numbering between the non-sintered ceramic and the sintered ceramic.

The feedthrough illustrated in FIG. 3B may also be obtained by brazing methods.

In certain particular cases, it may be advantageous that one or several of these pins be hollow for example in order to allow the device to be filled with neutral gas before hermetically sealing it, for example by laser welding.

According to the type of ceramic used, the temperatures required for co-sintering will be comprised between 1,200° C. and 1,700° C. and preferentially equal to 1,450° C. Relatively slow temperature raising ramps will have to be used in order to allow total removal of the organic components before attaining the temperature plateau at which the co-sintering annealing will take place. Thus, these ramps may be comprised between 0.1° C./min and 5° C./min, and preferentially equal to 1° C./min.

The ceramics used for producing hermetic feedthroughs are advantageously in alumina (Al₂O₃) or in Zirconia (ZrO₂) stabilized with yttrium oxide (Y₂O₃).

The thickness of the ceramics used for making the metal feedthroughs 301 and 302 is comprised between 10 μm and 1 mm, and preferentially equal to 500 μm.

Another hermetic feedthrough (not shown) consists in a combination of the structures shown in FIGS. 5A and 5B.

Thus, a through-via is made within a first body in non-sintered ceramic which is filled by means of an ink based on Pt for example.

A metal track is then deposited on the lower face of the non-sintered ceramic body.

A through-via is then made within a second body in non-sintered ceramic in which a metal pin will be positioned.

Both bodies as well as the metal pin and the platinum track are put into contact and co-sintered at a temperature comprised between 1,200° C. and 1,700° C., and preferentially equal to 1,450° C., in order to obtain a hermetic feedthrough ensuring electric continuity between the upper face of the first ceramic body and the lower face of the second ceramic body.

Thus structure has the advantage of gaining room inside the casing, while allowing contact to be easily resumed outside the casing.

The method according to the invention then consists of assembling hermetic feedthroughs, as illustrated in FIGS. 5A and 5B on the structures illustrated in FIGS. 3 and 4.

Depending on the materials used, various assembling methods may be used. Diffusion welding or brazing may notably be considered.

Thus, FIG. 6 illustrates the structure of FIG. 3, in the bulges of which have been assembled feedthroughs as illustrated in FIG. 5 for obtaining a feedthrough device according to the invention.

The obtained structure advantageously has a thickness of less than 1.5 mm and, preferably less than 500 μm.

FIG. 6 shows the electrical connection element of each feedthrough (the track 33 or the pin 35) partly passes through the opening 21 of a bulge 11.

Of course, FIG. 6 is only an exemplary embodiment and other hermetic feedthroughs may be integrated into the bulges 11.

Moreover, in the example illustrated in FIG. 6, the assembling of the feedthroughs 101 and 102 into the bulges 11 is achieved by means of a brazing joint 41.

Of course, the invention is not limited to this embodiment and other techniques may be used for achieving this assembling.

The brazing joint 41 may be of a highly diverse chemical nature depending on the targeted application.

Within the scope of an implantable biomedical application, one of the key points is the biocompatibility of the brazing joint 41. For example mention may be made of a joint based on titanium and nickel, a material known under the trade name of TiNi50, or a joint based on pure nickel. In this respect reference may be made to the document of Jiang “Development of ceramic to metal package for Bion microstimulator” (ProQuest Dissertations and Theses; Thesis (Ph.D.)—University of Southern California, 2005; Publication Number: Aal3196824; ISBN; 9780542410758; Source: Dissertation Abstracts International, Volume: 66-11, Section: B, page: 6104.; 135p.).

This brazing step has the purpose of hermetically sealing the substrate 10 to the hermetic feedthroughs 301 and 302, so as to obtain a hermetic feedthrough device.

The thickness of the brazing joint 41 will be comprised between 500 nm and 100 μm.

The hermetic feedthroughs 301 and 302 may be totally or partly integrated within the bulge 11.

The obtained intermediate product gives the possibility of retaining some flexibility of the metal substrate 10.

Indeed, the areas 51 have significant flexibility, while the areas 52 are rigid and give the possibility of thereby considerably reducing the stresses on the joint 41.

This gives the possibility inter alia of retaining a globally flexible substrate 10 while imposing no or very few mechanical deformations at the hermetic joints 41.

On this subject, by simplification, there is no distinction made in terms of numbering between the brazing joints used for brazing the ceramic body on the substrate 10 and the hermetic joints corresponding to the product of the brazing of the ceramic body on the substrate 10 via the brazing joint.

It is extremely important to retain the integrity of the joints all along the lifetime of the implantable system. In the opposite case, the electronic components present within the casing risk being rapidly deteriorated because of infiltration of biological fluids within the casing.

Advantageously, the ceramic 34 will be in intimate contact with the wall 13.

In the case when the ceramic would not be in direct contact with the wall 13, a material may be added in order to fill in the space between the ceramic 34 and the wall 13. This material may be a metal, but also a rigid polymer.

Further, as mentioned earlier, it is important that the surface area occupied by the aperture 21 be much smaller than the surface area of the base 14. Indeed, the contact surface area between the ceramic and the outer medium is then small and consequently, the ceramic will be mechanically protected by the base 14 and this, even when the ceramic thickness used in making these hermetic feedthroughs is small.

In order to further improve this mechanical protection, it is possible to add, at the non-protected portion, a biocompatible polymer 61 as shown in FIG. 7.

This polymer may be rigid or flexible, but preferentially rigid. It will give the possibility, in addition to the mechanical protection, of ensuring the isolation between the pin 35 and the metal track 33 and the metal substrate 10, at the opening 21.

This polymer 61 may also facilitate the connection of one or several electrode probes by being used as a guide and for maintaining the latter.

FIG. 7 illustrates the structure of FIG. 6, in a deformed position. In this position, the areas 51 located between the bulges 11 are deformed since they have significant flexibility. On the other hand, the areas 52 located at the bulges are not subject to any deformation, because of their rigidity, which allows reduction in the stresses on the joints 41.

Generally, by flexible structure is meant a structure which may be conformed to a diameter comprised between 3 and 12 cm, and preferably equal to 7 cm.

Reference is now made to FIG. 8 which illustrates a last step of the method according to the invention for obtaining a hermetic casing.

In this last step, a lid 71 is assembled to the substrate 10 of the structure illustrated in FIG. 6.

This lid 71 is preferably made in metal and notably in titanium.

The shape of the metal lid 71 is selected so that once it is attached to the substrate, the lid forms a cavity which may receive at least one portion of the electronic components.

The assembling of the lid 71 to the substrate 10 may be achieved with different techniques, notably laser welding 72 which gives the possibility of making welds in specific areas with localized heating.

The height of the lid will be less than 3 mm and advantageously less than 2 mm. It is preferentially comprised between 500 μm and 1 mm.

Having a casing which has some flexibility is an essential criterion for the new generations of implantable devices. This will give the possibility of implanting it in any areas, the casing being able to be conformed to the area to be therapeutically treated, considering the flexibility.

The obtained encapsulation casing is a thin casing, i.e. its global thickness is less than about 3 mm.

Reference is now made to FIG. 9 which illustrates two alternatives for applying the step of the method illustrated in FIG. 6.

The alternative illustrated in FIG. 9A is designed for increasing the rigidity of the area of the substrate where the feedthrough is assembled and also for increasing the reliability of this feedthrough.

As shown by FIG. 9A, the feedthrough 801 which is hermetically assembled on the substrate 10 is of the type of the feedthrough 302 illustrated in FIG. 5B. It thus includes a ceramic body 34 which is crossed by a metal pin 35.

At one of its ends, the body 34 has an outgrowth 340 giving it the shape of a T.

Consequently, the feedthrough 801 may be assembled to the substrate 10 in two distinct areas.

FIG. 9A shows that the feedthrough 801 is assembled at the base 14 of the bulge 11 by means of a brazing joint 41. It is also assembled to the surface 100 of the substrate 10, opposite to the surface 101 including the bulge, via the brazing joint 802. The latter is provided between the outgrowth 340 and the surface 100.

The brazing joints 41 and 802 may be of an identical or different nature.

Preferentially they will consist of nickel and titanium.

In the example shown in FIG. 9A, both brazing joints 41 and 802 will have a ring structure.

Thus, the brazing joint 41 will be placed within the bulge 11, while the brazing unit 802 will be placed at the periphery of the bulge 11.

The reliability of the casing is increased as compared to that of the structure illustrated in FIG. 8, without reducing its flexibility. Indeed, in the case of a failure of one of the two brazing joints 41 or 802, the casing will retain its air tightness.

The alternative embodiment illustrated in FIG. 9B also has the purpose of increasing the reliability of the assembly between the feedthrough and the substrate 10 illustrated in FIG. 3A.

The feedthrough 302 assembled in the substrate 10 has already been described with reference to FIG. 5B.

The hermetic assembly is made by means of a brazing joint 91 which has a three-dimensional structure. This structure gives the possibility of increasing the assembling surface between the feedthrough 302 and the substrate 10.

By having the hermetic feedthrough found within a bulge 11 gives the possibility of applying pressure forces during the brazing annealing, both parallel to the plane of the substrate 10 (at the side wall 13) and perpendicularly to the plane of the substrate 10 (at the base 14 and at the upper face of the ceramic). Further, because of the flexibility of the substrate 10, it is easy to maintain an intimate contact between the wall 13 and the hermetic feedthrough, which gives the possibility of producing a quality assembly.

Further, when the bulges have a conical shape, it is possible to apply a pressure force during the brazing annealing simultaneously at the wall 13 and at the base 14, by exclusively applying a pressure force parallel to the axis of the pin 35.

Reference is now made to FIG. 10 which illustrates three alternative embodiments of the substrate illustrated in FIG. 3. These three alternatives are designed so as to increase the rigidity of the area of the substrate where the feedthrough is assembled.

Thus, FIG. 10A shows a substrate 10 including a bulge 11 with a side wall 13 and a base 804 which includes an over-thickness, with respect to the base 14 of the substrate illustrated in FIG. 3.

In the bulge, a feedthrough 302 is assembled, of the type illustrated in FIG. 5B.

This base 804 with increased thickness gives the possibility of ensuring a more significant resistance to impacts, without reducing the flexibility of the substrate 10.

Thus, the alternative embodiment of FIG. 10A gives the possibility of increasing the rigidity of the area where the feedthrough is assembled, improving its mechanical strength and reducing the risks of breaking the ceramic making up the feedthrough.

Further, by locally having an over-thickness at the wall 804 may prove to be useful in the case of diffusion of one or several elements making up the brazing joint 41 within the substrate 10.

Indeed, in the particular case of a brazing joint 41 based on Ni or on TiNi50 and of a substrate 10 based on titanium, the Ni may diffuse over several tens of microns within the titanium. Accordingly, if, at the brazing joint, the thickness of the substrate is insufficient, the nickel will be able to diffuse as far as the end opposite to the one where the brazing takes place thereby causing a risk of brazing the substrate holder (not shown on the various diagrams) being used inter alia for applying a certain force on the sample during the brazing.

It should be noted that the diffusion length of the elements making up the brazing joint 41 within the substrate 10 will depend on several parameters including the brazing temperature.

In the alternative illustrated in FIG. 10B, it is the side wall 803 of the bulge 11 which has an over-thickness, the base 14 of the bulge 11 being similar to that of the bulge illustrated in FIG. 3.

This alternative also gives the possibility of increasing the rigidity of the area where the feedthrough 302 is assembled, by means of this lateral over-thickness.

The alternative illustrated in FIG. 100 illustrates a substrate 10 with a bulge 11, for which the side wall 13 and the base 14 are similar to those of the bulge illustrated in FIG. 3.

On the other hand, an over-thickness 815 is provided on the wall 100 of the substrate opposite to the wall 101 including the bulge 11.

This over-thickness 815 has a ring shape and extends the inner face of the side wall 13.

In this alternative embodiment, a feedthrough 801 as illustrated in FIG. 9A is assembled.

Thus, this feedthrough 801 is assembled to the substrate 10 via two brazing joints. The first joint 41 is located between the feedthrough and the base 14 of the bulge 11, while the other joint 814 is located between the outgrowth 340 of the feedthrough and the upper face of the over-thickness 815.

This alternative further gives the possibility of rigidifying the obtained structure, of reducing both the stresses on the joint 41 because of the bulge 11 but also on the joint 814 because of the over-thickness 815.

Reference is now made to FIG. 11 which illustrates four alternatives (FIGS. 11A to 11D) for assembling a feedthrough on a support of the type illustrated in FIG. 4, and another more specific alternative (FIG. 11E).

In the alternative illustrated in FIG. 11A, a feedthrough 302, of the type illustrated in FIG. 5B, is positioned inside an over-thickness 15 of the substrate 10.

For this, the over-thickness 15 has a suitable housing defined by a side wall 150 and a bottom 151.

A brazing joint 41 is provided between the feedthrough 302 and the bottom 151 of the housing. The over-thickness 15 gives the possibility of increasing the rigidity of the area where the feedthrough is assembled.

This alternative gives the possibility of avoiding the presence of protruding portions on the external face of the casing which may be obtained from the structure illustrated in FIG. 11A. This gives the possibility of increasing the compatibility of the casing with biological tissues, in contact with it.

The alternative illustrated in FIG. 11 B consist of assembling a feedthrough of the type illustrated in FIG. 5B, on the upper face 152 of the over-thickness 15 present on the substrate 10.

Considering the width of the over-thickness, the body 34 of the feedthrough 302 has larger dimensions than those of the body 34 illustrated in FIG. 11A.

Moreover, a brazing joint 41 is provided between the feedthrough 302 and the upper face 152 of the over-thickness 15.

This alternative has the effect of increasing the rigidity of the area where the hermetic feedthrough is assembled by means of the over-thickness 15.

Further, this gives the possibility of reducing the risks of failure of the ceramic making up the hermetic feedthrough. Indeed, the over-thickness allows protection of the ceramic against possible mechanical impacts.

Finally, this also allows a reduction in the thickness of the protrusions on the external face of the casing in contact with the biological tissues.

In the alternative illustrated in FIG. 11C, the body 34 of the feedthrough 302 is brazed to the inner wall 156 of the over-thickness 15 via a brazing joint 41. This allows a lateral brazing of the body 34 to the over-thickness 15 which will then have a reinforced mechanical strength as compared with the structure shown in FIG. 6.

The alternative illustrated in FIG. 11D allows simplification of the application. The body 34 of the feedthrough 302 illustrated in FIG. 5B is brazed beforehand to the wall 153 of the over-thickness 15 via a brazing joint 41, before hermetically assembling the assembly 155 (comprising the body 34, the pin 35, the hermetic joint 41 and the over-thickness 15) to the substrate 10 via a hermetic joint 154.

This assembly may for example be made by laser welding.

Precautions will have to be taken in order not to deteriorate the hermetic joint 41. The hermetic joint 154 may thus be shifted by a distance comprised between 100 μm and 1 mm, preferentially 500 μm, with respect to the hermetic joint 41.

As an alternative of the embodiment illustrated in FIG. 11D, FIG. 11E illustrates another method for assembling the assembly 155 on a substrate 10.

Thus, in order to more easily position the assembly 155 with respect to the substrate 10, it is possible to put this assembly 155 within a bulge 11. A hermetic joint 156 between the over-thickness 15 and the base 14 may be achieved by laser welding.

It should be noted that it is also possible to produce a hermetic joint (not shown) between the outer wall 157 of the over-thickness 15 and the wall 13 of the bulge 11.

Finally reference is made to FIG. 12 which illustrates a substrate 12 which has the shape of a thick but structured sheet.

Thus, thinner areas 120 are made, by removal of material for example. Protruding areas 121 with respect to substrate 12 are then defined. They form an over-thickness.

Thus, the substrate 12 is relatively flexible by the presence of these areas 120, which will give global flexibility to the casing which will be obtained from the substrate 12.

The depth of these areas 120 may be comprised between 5 and 95% of the thickness of the sheet, preferentially 60%.

Thus, if a substrate consisting of a titanium sheet with a thickness of 250 μm is taken as an example, it will be possible to produce areas 120 with a depth comprised between about 12 μm and 235 μm.

Also, the number of areas 120 will depend on the desired flexibility.

Indeed, the larger the number of areas 120 and the more flexible will be the substrate 12.

Thus the percentage of the surface area occupied by the areas 120 may be comprised between 5% and 95%, preferentially 60%.

In the area 121 located between two areas 120, a through-opening 21 is made. This opening may be made before or after forming the area 121.

On the area 121, a feedthrough 302 is assembled of the type illustrated in FIG. 5B, by means of a brazing joint 41, so that the pin 35 passes through the aperture 21.

Thus, like in the examples described earlier, the area of the substrate in which the feedthrough is assembled is rigidified, while retaining the global flexibility of the substrate.

FIG. 13 illustrates another alternative which gives the possibility of avoiding the brazing of the substrate holder during the formation of the brazing joint 41.

FIG. 13 illustrates a ring in ceramic 130 (for example in alumina (Al₂O₃) or in Zirconia (ZrO₂) stabilized with yttrium oxide (Y₂O₃)) which is placed outside the bulge 11.

This ring 130 is then placed against the base 14 of the bulge 11.

It may be placed outside an over-thickness 15 (substrate-holder side) for a substrate of the type illustrated in FIGS. 4A and 4B

During the brazing, if the elements making up the brazing joint 41 were to diffuse as far as this ceramic ring (one of the possible diffusion directions of the brazing components is illustrated by an arrow in FIG. 13), this ring 130 would then be brazed to the bulge 11 or to the over-thickness.

This ring will then be an integral portion of the device and avoids brazing of the substrate holder.

The reference markings inserted after the technical characteristics appearing in the claims have the sole purpose of facilitating the understanding of the latter and cannot limit the scope thereof.

1. A feedthrough device especially for a medical implant system comprising:
a generally flexible substrate locally comprising at least one means for stiffening the substrate, said at least one stiffening means having a through-opening; and
at least one hermetic feedthrough comprising an electrical connection element, said feedthrough being hermetically assembled to a stiffening means such that said elctrical connection element passes through the through-opening.

2. The feedthrough device according to claim 1, wherein said substrate includes a metal sheet.

3. The feedthrough device according to claim 1, wherein said at least one stiffening means includes a protruding portion with respect to the surface of the substrate.

4. The feedthrough device according to claim 3, wherein the protruding portion is a bulge or an over-thickness.

5. The feedthrough device according to claim 1, comprising at least one brazing joint between a feedthrough and a stiffening means.

6. The feedthrough device according to claim 5, wherein said at least one brazing joint has a three-dimensional structure.

7. The feedthrough device according to claim 5, wherein the feedthrough has a shape of a T, a second brazing joint being present between an outgrowth and the substrate.

8. A method for manufacturing a feedthrough device according to claim 1, comprising:
(a) producing a generally flexible substrate with at least one stiffening means and at least one through-opening in said at least one stiffening means,
(b) providing at least one hermetic feedthrough including an electric connection element, and
(c) hermetically assembling a feedthrough to said at least one stiffening means, so that the electrical connection element of said feedthrough partly passes into said through-opening.

9. The method according to claim 8, wherein the step (a) includes producing at least one protruding portion with respect to the substrate.

10. The method according to claim 9, wherein the step (a) includes a local deformation of the substrate in order to form at least one bulge.

11. The method according to claim 9, wherein the step (a) includes providing material for locally forming at least one over-thickness.

12. The method according to claim 9, wherein the step (a) includes removing material in at least two areas of a substrate in order to form at least one over-thickness between these areas.

13. The method according to claim 8, wherein step (c) is achieved by at least one brazing joint.

14. The method according to claim 8, comprising, after step (c), depositing a polymeric material, in said at least one through-opening.

15. The method according to claim 13, comprising, between step (b) and step (c), positioning of a ceramic ring against said at least one stiffening means and on the substrate holder side.

16. A hermetic casing including a feedthrough device according to claim 1 and a lid hermetically sealed to the feedthrough device, for encapsulating a medical device.
